(19) United States
(12) Patent Application Publication
(10) Pub. No.: US 2023/0005575 A1
(43) Pub. Date: Jan. 5, 2023

(54) SYSTEMS AND METHODS FOR RECOMMENDING MEDICAL TESTS

(71) Applicant: KONINKLIJKE PHILIPS N.V., EINDHOVEN (NL)

(72) Inventors: Jörn Borgert, Hamburg (DE); Thomas Netsch, Hamburg (DE); Thomas Erik Amthor, Hamburg (DE)

(21) Appl. No.: 17/779,786

(22) PCT Filed: Nov. 20, 2020
(86) PCT No.: PCT/EP2020/082796

§ 371 Date: May 25, 2022

Related U.S. Application Data
(60) Provisional application No. 62/940,390, filed on Nov. 26, 2019.

(51) Int. Cl. G16H 10/60; G16H 50/20

(52) U.S. Cl. CPC G16H 10/60; G16H 50/20; G16H 10/20

(57) ABSTRACT

An apparatus (10) for recommending medical tests for a patient includes at least one electronic processor (20) programmed to retrieve patient data stored in a database (32); compare the patient data with guidelines (34) for a plurality of medical tests and identify at least one recommended medical test of the plurality of medical tests based on the comparison; and output, on at least one display device (24), a recommendation (40) for the patient to undergo the at least one recommended medical test.

### FIELD

The following relates generally to the health management arts, patient screening arts, patient medical testing arts, patient medical testing recommendation arts, and related arts.

### BACKGROUND

There are a wide range of medical screening tests that can be performed on patients, which can provide early detection of serious medical conditions which enhances health outcomes; or, conversely, can rule out serious medical conditions thereby providing the patient with peace of mind. Screening tests are usually performed on the basis of certain guidelines (e.g., a colonoscopy is recommended for patients over the age of fifty) and require various inputs from blood tests, histopathology tests, dedicated medical examinations (e.g. colonoscopy), and so forth. Usually, a physician prescribes a screening test for a patient on the basis of the guidelines and also on the basis of the physician's specific knowledge of the patient's pre-existing medical conditions, and the physician's own experience.

This approach has certain disadvantages. Physicians usually have a high case load, and may fail to recognize and prescribe a useful screening test for a patient. When a screening test is prescribed, the patient may undergo duplicative lab work to collect the patient data for the test. For example, a liver screening may require certain blood tests, but even if the patient has already recently had those blood tests, they may nonetheless be rescheduled for the liver screening. Another aspect of this problem is that, if a patient has certain lab work performed, this may provide most of the patient data to perform a useful screening test, yet the physician may not recognize this situation and hence may fail to prescribe the screening test, even though it may require collecting only a small amount of additional patient data (or perhaps no additional data collection at all).

The following discloses certain improvements to overcome these problems and others.

### SUMMARY

In one aspect, an apparatus for recommending medical tests for a patient includes at least one electronic processor programmed to retrieve patient data stored in a database; compare the patient data with guidelines for a plurality of medical tests and identify at least one recommended medical test of the plurality of medical tests based on the comparison; and output, on at least one display device, a recommendation for the patient to undergo the at least one recommended medical test.

In another aspect, a non-transitory computer readable medium stores instructions executable by at least one electronic processor to perform a method of recommending medical tests for a patient. The method includes: retrieving patient data stored in a database; comparing the patient data with guidelines for a plurality of medical tests; identifying multiple recommended medical tests of the plurality of medical tests based on the comparison; ranking the multiple recommended medical tests based on one or more ranking criteria; and outputting, on at least one display device, a list of multiple recommendations for the patient to undergo the multiple recommended medical tests according to the one or more ranking criteria.

In another aspect, a method for recommending medical tests for a patient includes: retrieving patient data stored in a database; comparing the patient data with guidelines for a plurality of medical tests; identifying at least one recommended medical test of the plurality of medical tests based on the comparison; generating a user interface showing at least one of: a recommendation of the at least one recommended medical test, currently available patient data stored in the electronic medical record database, and additional data needed to complete the at least one recommended medical test; and transmitting at least the recommendation to one or more of the patient, a doctor of the patient, and an insurance company of the patient.

One advantage resides in providing an overview for a physician to view existing tests and diagnostics and determine any potential follow-up procedures.

Another advantage resides in using existing test results of a patient for use in future tests for the same patient.

Another advantage resides in extracting medical claims from existing tests results to recommend additional tests to be performed.

Another advantage resides in maximizing usage of a blood or tissue sample taken from a patient.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative screening test recommender apparatus for recommending screening tests for a patient in accordance with the present disclosure.

FIG. 2 shows example flow chart operations performed by the apparatus of FIG. 1.

FIG. 3 shows an example of a recommender engine of the apparatus of FIG. 1.

### DETAILED DESCRIPTION

In some embodiments disclosed herein, a recommender engine is provided for recommending screening tests, identifying patient data already available for the screening tests, and identifying the additional lab work needed. The recommender engine mines the patient's electronic medical (or health) record and compares the guidelines for various screening tests against the patient's pre-existing medical conditions and the available patient data. Based on this comparison, various screening tests are identified as potentially useful for the patient. For each potential screening test, the recommender engine determines what patient data may already be available for use in the screening test, and what patient data still needs to be collected and the lab work needed to collect it. As patient data becomes less reliable over time, the age of the patient data may be taken into account and older patient data discarded (e.g. if it is too old to be considered reliable) and the retained patient data annotated with its acquisition date.

The recommender engine scores each potential screening test based on criteria such as the clinical guideline for the screening test itself (e.g., is this test highly recommended for all patients at this patient's age?), and any pre-conditions or other patient characteristics (e.g., a liver test may be more highly ranked if the patient is indicated as being a relatively heavy alcohol user; or a screening test may be ranked higher if the patient is of an ethnicity for which the condition being tested is more prevalent). Another possible ranking factor may be how much additional lab work is required to perform the test—if only a single lab visit will enable performing the screening test then it may be ranked higher. Other possible ranking factors may relate to health insurance coverage of the patient; for example, if the patient's health insurance will fully reimburse for the cost of performing the screening test then it may be ranked higher. If the patient has had the screening test in the past, then the scoring may take into account the recommended time interval for the screening test to be repeated. For example, if a screening test is recommended to be repeated every five years, then if the patient has had the screening test within the last five years this screening test is not recommended, or the prior test is taken into account in scoring (e.g., if the guideline calls for the test every 5 years and the last prior test was 4 years ago it may still be recommended, but at lower score).

In a further aspect, if sufficient patient data is available to perform a certain screening test immediately, by processing already-available patient data, then the screening test is performed and the test result generated.

In yet another aspect, the recommender system may be invoked in conjunction with scheduling of an ordered hematology lab test, histopathology lab test, or other physician-ordered patient test which entails obtaining a blood or tissue sample from the patient. The recommender system may then assess whether the drawn blood or tissue sample could be used for an additional screening test (in addition to the scheduled patient test), and, if so, a recommendation may be made to perform the additional screening test. In a variant approach, the recommender system may also estimate the quantity of blood or tissue sample required to perform both the scheduled patient test and the additional screening test, and if the quantity of blood or tissue to be extracted in accord with the physician-ordered patient test is insufficient to also perform the additional screening test, then the recommendation may also specify the amount of blood or tissue that would need to be extracted to perform both tests.

Based on the foregoing information, a user interface (UI) presents potential screening tests to the physician, and optionally also to the patient, and optionally also to the patient's insurance company. The scope of recommended screening tests, and the level of detail given for each recommended screening test, may be tailored to the recipient. In general, the patient's physician is given the most information, contemplated to include the recommended screening tests, date of the last time the screening test was administered to the patient (if applicable), the already available patient data for the test annotated by ages of the data, and the additional patient data required to perform the screening test and the lab work needed to collect it. The information provided to the physician may also include the guideline recommendations for triggering the test (e.g., age threshold, preexisting conditions that for which the screening test is recommended).

To meet regulatory requirements and the like, the recommender system typically cannot actually order a screening test to be performed. Rather, the physician may override the recommender engine by rejecting a recommended screening test, or indicating it should be revisited in one year (or when the patient reaches age 50, some other chosen time interval). Similarly, the physician may reject use of aged patient data that was not automatically discarded by the recommender system. In some embodiments, the physician may select and approve the screening test at which point the recommender system accesses a hospital scheduling system to automatically schedule the test.

In other embodiments, results of any screening test that is automatically performed by the recommender system by processing already-available patient data are also presented to the physician, and are entered into the patient's electronic record upon review and approval of the physician. Furthermore, the recommender system dynamically updates its recommendations, so that for example if it detects that a recommended screening test has now been performed and the results added to the patient's electronic record then that screening test recommendation is removed.

By contrast, the patient's UI is contemplated to provide much less information, and optionally also less control over the list of recommended screening tests. Typically, the patient's UI may only list those recommended screening tests whose score is above a threshold, possibly along with information on already-available patient data and the lab work needed to collect any missing patient data. The patient generally cannot reject a test in the patient's UI (rather, the patient should discuss with the physician and the latter can reject the test in consultation with the patient). Any automatically performed screening test results are not presented to the patient via the patient's UI. Alternatively, if applicable regulations require that the patient have the ability to reject a test, and/or require that the patient receive the test results, then the patient's UI can provide these capabilities. It is contemplated to provide different patient UI features in different regulatory jurisdictions in order to ensure regulatory compliance. The insurance company's UI is likely to be similar to that of the patient, except that more information may be excluded in order to comply with HIPAA or other applicable patient privacy regulations, and no control capability is provided.

Typically, a set of M patient data are required to perform a given screening test. However, in practice this set M is often not rigid. There may be a minimum set of patient data required to perform the screening test, but additional patient data beyond that minimum may provide higher confidence in the test result. In such cases, the minimum patient data for performing the screening test and also the additional patient data that would enhance confidence may be presented to the physician. Where a test result is automatically generated by processing already-available patient data, a confidence metric may also be provided and, in case of a positive test result, it may be recommended to obtain the additional patient data in order to enhance confidence.

In other optional aspects disclosed herein, the recommender engine may be incorporated into a lab work ordering system to maximally utilize collected blood or tissue samples, or maximally acquire data during an invasive procedure such as a colonoscopy. For example, if the physician orders a complete blood count (CBC), then at the time of entering this order the recommender system may determine whether additional blood work could be performed on the blood vial (or vials) acquired for the CBC in order to provide sufficient patient data to perform a recommended screening test. If so, then the recommender system may recommend performing the additional blood work using blood collected for the already-ordered CBC. Optionally, this may take into account the amount of blood (or tissue) to be acquired for the ordered lab work and determine whether additional blood (or tissue) would be needed for performing the recommended additional lab work, and, if so, recommend collecting the additional blood or tissue.

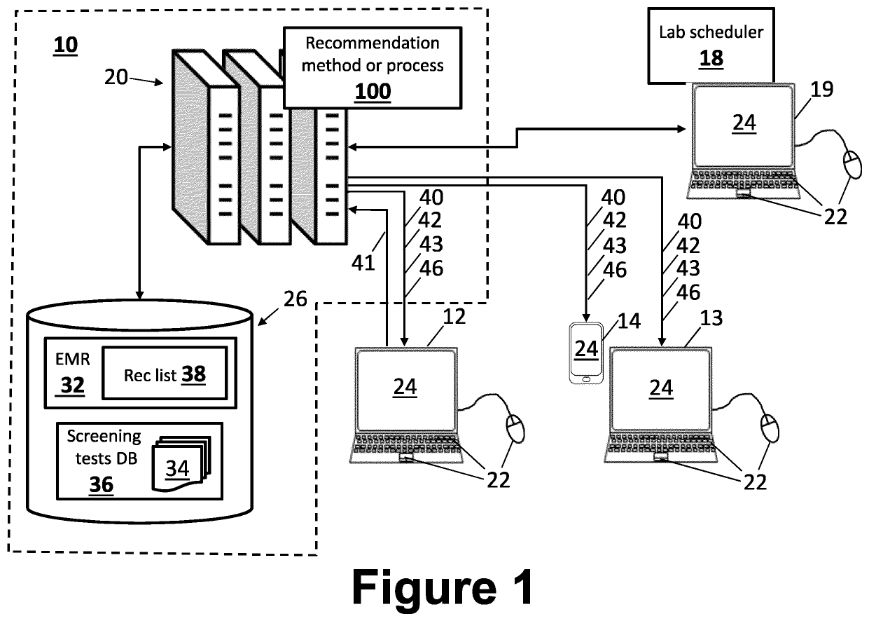

With reference to FIG. 1, an illustrative screening test recommender apparatus 10 is implemented on an electronic processor 20, such as a server computer or illustrative multiple server computers 20 (e.g., a server cluster or farm, a cloud computing resource, or so forth), which implements a medical (e.g. screening) tests recommender method 100 as disclosed herein. To perform the recommender method 100, the electronic processor 20 accesses at least one non-transitory storage medium 26 that stores a screening (or, medical) tests database 36, and at least one database 32 storing patient medical records. The illustrative database(s) 32 are an electronic medical record (EMR) 32; however, other nomenclatures may be used (e.g., electronic health record, EHR), and/or the database(s) 32 may include domain specific patient records databases such as a Picture Archiving and Communication System (PACS) database and/or a Radiology Information System (RIS) which stores medical imaging-specific patient data, a cardiovascular information system (CIS or CVIS) which stores patient data collected and maintained by the patient's cardiologist and/or a cardiology department, and/or so forth. As will be discussed, the medical tests recommender method 100 maintains a list 38 of the medical tests that are recommended for a patient. The medical tests recommender method 100 pushes this list 38, or a portion thereof, to a physician's user interface (UI) device 12, e.g., UI device 12 may be the physician's desktop computer, a tablet or notebook computer owned by or assigned to the physician, a cellphone owned by or assigned to the physician, various combinations thereof, and/or so forth.

The medical tests recommender method 100 pushes a recommendation 40 for the patient to undergo the at least one of the recommended medical tests to various user interface (UI) devices 12, 13, 14. For example, in the illustrative example, a screening test recommendation 40 may be pushed to a physician's UI device 12 (e.g., the physician's desktop computer, a tablet or illustrative notebook computer 12 owned by or assigned to the physician, a cellphone owned by or assigned to the physician, various combinations thereof, and/or so forth), and/or to a patient's UI device 13, 14 (e.g., the patient's desktop computer, a tablet or illustrative notebook computer 13 owned by or assigned to the patient, an illustrative cellphone 14 owned by or assigned to the patient, various combinations thereof, and/or so forth), and/or to an insurance company's UI (not shown). The physician's UI device 12 may also be used to send feedback 41 back to the recommender method 100, such as a rejection of a recommended test or instructions to postpone a recommended test for a certain time period. It may be noted that FIG. 1 illustrates UI devices 12, 13, 14 for the patient and the patient's physician. However, more generally, the medical tests recommender method 100 preferably services a large group of patients (e.g., all patients being treated by the physician or a physicians' group, all patients being insured by a particular medical insurance provider, or so forth). Hence, there may in general be corresponding UI devices 12, 13, 14 for each patient pushing recommendations 40 specific to each patient. Likewise, a patient-specific tests recommendation list 38 is suitably maintained by the recommender method 100 for each patient.

In some embodiments, the medical tests recommender method 100 also interacts with a hematology, histopathology, or other lab scheduler 18 that schedules patient tests that involve drawing a blood sample from the patient or extracting a tissue sample from the patient. For example, the lab scheduler 18 may be implemented on a server or a workstation 19 that is used to schedule the patient for a physician-ordered Complete Blood Count (CBC) or other physician-ordered patient test requiring a blood draw or tissue sample. The recommender method 100 receives information about this scheduled physician-ordered patient test, optionally in real time, and determines whether any of the medical tests on the list 38 of medical tests (excluding the physician-ordered patient test, if it is on the list) could be performed using the blood or tissue sample extracted for the physician-ordered patient test. If so, then the recommendation 40 may include a recommendation to perform the additional test from the list 38 that can be completed using the blood or tissue already scheduled for extraction in order to perform the physician-ordered test. Optionally, this recommendation may additionally or alternatively be sent to the scheduling workstation 19, and/or to the patient.

The various UI devices 12, 13, 14, 19 may include typical components, such as an electronic processor (e.g., a microprocessor), at least one user input device (e.g., a mouse, a keyboard, a trackball, and/or the like) 22, and a display device 24 (e.g. an LCD display, plasma display, cathode ray tube display, and/or so forth). The electronic processor 20 is implemented as a recommender engine performing the recommender method 100, and is operatively connected with memory 26 embodied as one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage accessible by the server computer(s) 20, an internal hard drive (e.g., if the electronic processor 20 is implemented on a desktop computer rather than on the illustrative server(s) 20), various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20 to perform the recommender method 100.

The at least one electronic processor 20 is configured as described above to perform the recommender method or process 100 for recommending one or more medical tests for a patient. The non-transitory storage medium 26 stores instructions which are readable and executable by the at least one electronic processor 20 to perform disclosed operations including performing the method or process 100. In some examples, the method 100 may be performed at least in part by cloud processing.

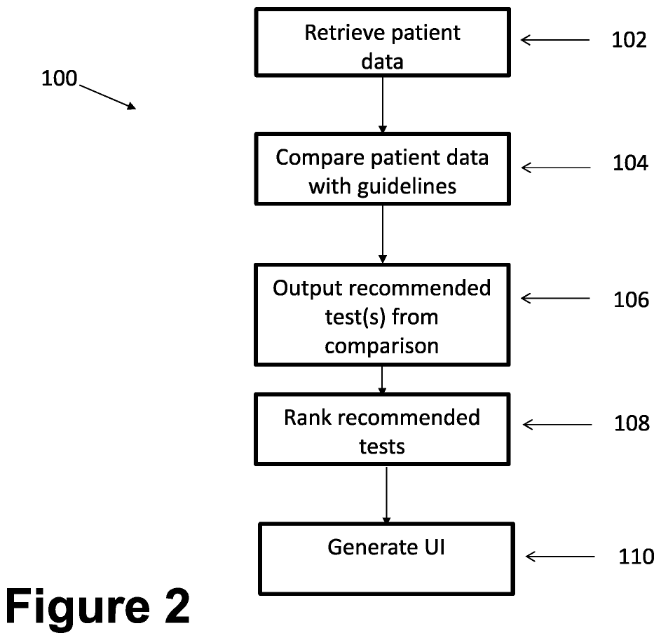

With reference to FIG. 2, and with continuing reference to FIG. 1, an illustrative embodiment of method 100 is diagrammatically shown as a flowchart. At an operation 102, the at least one electronic processor 20 is programmed to retrieve patient data stored in the database 32. The database 32 can, as previously mentioned, be any suitable database, such as, for example, an electronic medical record (EMR) database, an electronic health record (EHR) database, a Picture Archiving and Communication System (PACS) database, a Radiology Information System (RIS), and so forth. In some embodiments, a physician can determine acquisition date(s) of items of the patient data, and remove any item of the patient data having an acquisition data that is older than a predetermined time threshold. For example, if a patient is recommended to have a colonoscopy every 3 years, then any items for a patient including a colonoscopy obtained more than 3 years ago can be discarded. In another approach, the predetermined time threshold may be a configuration parameter of the recommender method 100, and the physician may optionally be able to override this (default) time threshold. It should be noted that the predetermined time threshold may be different for different types of patient data, as different types of patient data may be considered “stale” at different ages, and some patient data (e.g. the patient's ethnicity) may never be expected to change.

At an operation 104, the at least one electronic processor 20 is programmed to compare the patient data with guidelines 34 of the plurality of medical tests stored in a screening or medical tests database 36 and identify at least one recommended medical test based on the comparison. The guidelines 34 and other data of the plurality of medical tests database 36 can be stored in the non-transitory computer readable medium 26. For example, the guidelines 34 for various screening tests are compared against the patient's pre-existing medical conditions and the available patient data retrieved from the database(s) 32. Based on this comparison, at least one recommended screening test is identified as potentially useful for the patient.

At an operation 106, the at least one electronic processor 20 is programmed to send the recommendation 40 to one or more of the UI device(s) 12, 13, 14 for display on the display device 24 to display an output of the recommendation 40 for the patient to undergo the at least one recommended medical test 38. The computational and control processing involved with displaying the recommendation 40 may be variously shared between the server(s) 20 and the UI device 12, 13, 14. For example, a workstation UI device 12, 13 may be running a web browser which connects with a webpage hosted by the server(s) 20, and the browser running on the UI device 12, 13 receives web page content including the recommendation 40 and displays the web page content on the display device 24. In the case of a mobile device such as the illustrative cellphone 14, the mobile device may run an application program (“app”) that receives the recommendation 40 and locally constructs the presentation of the recommendation shown on the display device 24. These are merely illustrative examples.

In some embodiments, the comparing operation 104 includes determining missing information that is missing from the patient data that, if available, would enable execution of the at least one recommended medical test. In this embodiment, the output operation 106 includes outputting an identification 42 of the missing information that, if available, would enable execution of the at least one recommended medical test.

In other embodiments, the comparing operation 104 includes identifying an executable medical test of the plurality of medical tests in the screening tests database 36 based on the comparison for which the available information is sufficient to perform the medical test using the already-available patient data. For example, if a patient recently had blood test results taken for one medical test that can be used for a screening test of on the list 38, then that screening test on the list is performed using the available blood test results to generate a screening test result 43. The output operation 106 to the UI device(s) 12, 13, 14 then includes displaying the test result 43 on the display device 24. This approach is suitable when the screening test can be performed algorithmically based on patient data, without reliance upon subjective analysis or interpretation of patient data. For example, the standard Body Mass Index (BMI) screening test for obesity can be performed algorithmically using as inputs only the patient height and weight; hence, a BMI screening can be performed automatically upon receipt in the patient record of recent (i.e. not stale) patient height and weight measurements. By contrast, a screening test such as a mammography screening that requires expert interpretation of medical images by a clinician typically cannot be performed algorithmically (although in some cases automated image analysis via an artificial intelligence algorithm might be used to perform such a screening test algorithmically). The algorithms for performing screening tests are suitably stored in the screening tests database 36 for those tests that can be performed algorithmically.

Optionally, the at least one electronic processor 20 is programmed to generate a confidence value for the test result 43 based at least on the available information in the patient data. From this, the at least one electronic processor 20 is programmed to determine supporting information that is missing from the patient data that, if available, would increase the confidence value. The output operation 106 then includes a recommendation 46 to obtain the supporting information to increase the confidence value. This aspect can be implemented in other ways, some of which do not require computing a numerical confidence value. For example, the screening tests database 36 may store, for a given screening test, a list of patient data for performing the test and a list of supporting patient data that, if available, would increase confidence in the result. In this case, the output operation 106 would include a recommendation 46 to obtain the listed supporting information to increase the confidence in the test result.

In an optional operation 108, when multiple tests are recommended based on the comparing operation 104, the at least one electronic processor 20 is programmed to rank the multiple recommended medical tests 38 based on one or more ranking criteria. The one or more ranking criteria can include, for example: (i) one or more clinical guidelines 34 for each of the recommended medical tests 38 (e.g., whether an age or gender of the patient requires certain test to be prioritized over other tests); (ii) a pre-conditions or characteristics of the patient identified from the retrieved patient data (e.g., whether a patient or a patient's family has a history of, for example, diabetes or heart disease); (iii) additional lab work required to perform each of the multiple recommended medical tests based on the retrieved patient data (e.g., blood work, tissue samples, procedures such as colonoscopies, and so forth); (iv) an insurance reimbursement level for each test based on the retrieved patient data (e.g., how much an insurance company will cover the costs of the test(s)); and (v) a patient already having undergone one of the tests based on the retrieved patient data; among other ranking criteria. These are merely illustrative examples, and are not intended to limit the types of potential ranking criteria. From the ranking criteria, the at least one electronic processor 20 is programmed to generate a corresponding score for each criteria for each recommended test 38. A ranked list of the medical tests 38 based on the scores can be displayed on the display device 24, in, for example, descending order of the scores for each tests. Alternatively, only a top-N ranked list can be displayed (i.e., only a top-ranked N tests displayed).

Optionally, the at least one electronic processor 20 is programmed to generate the recommendation 40 with information about the recommended medical test(s) 38, including, for example, the recommended medical test(s), a scope of each recommended medical test, a level of detail for the at least one recommended medical test, currently available patient data stored in the database 32, and the additional data 42 needed to complete the at least one recommended medical test. These are merely illustrative examples.

The recommendation 40 can be transmitted to a user device 12 operable by the physician (e.g., a workstation, a laptop, a mobile device such as a smart phone or tablet, and so forth). In some embodiments, the physician can provide a user input 41 (e.g., via a mouse click, keystroke, finger swipe, and so forth) indicating a rejection of one or more of the recommended medical tests 38. For example, the physician can reject a recommended medical test 38 (e.g., a diabetes screening) if the physician knows that the patient recently had such a screening. In another example, the physician can reject a recommended medical test 38 if the patient knows that the test is not suitable for the patient (e.g., a test for a medical condition that would manifest in a long term time period that is recommended for a patient who has terminal cancer). The at least one electronic processor 20 is programmed to receive the input 41 from the physician indicating a rejection of at least one of the recommended tests 38, and update the list of medical recommended screening tests 38 to remove any rejected test(s). The updated ranked list 38 of tests can be updated automatically on the physician's user device, or an updated UI can be sent to the user device.

The recommendations 40 can also be pushed to a UI device of another party, such as to a UI device 13, 14 of the patient or a UI device (not shown) of the patient's insurance company. The information sent to the patient and/or the insurance company includes less information than the UI sent to the physician. The information sent to the patient can list recommended medical tests whose score is above a threshold, possibly along with information on already-available patient data and the lab work needed to collect any missing patient data. The patient generally cannot reject a recommended test—rather, the presented UI may preferably suggest that the patient discuss the recommended test with the patient's physician. The insurance company's UI is likely to be similar to that of the patient, except that more information may be excluded in order to comply with HIPAA (in the United States) or other applicable patient privacy regulations.

In another optional aspect, the apparatus 10 can comprise or interact with a laboratory procedure scheduling system 18 that (for example) includes the workstation 19 as described above. In this example, the at least one electronic processor 20 is programmed to schedule a physician-ordered laboratory procedure that requires collecting a blood or tissue specimen from the patient. The at least one electronic processor 20 is then programmed to determine missing information that is missing from the patient data (i.e., retrieved from the database 32) and that, if available, would enable execution of the at least one recommended medical test (e.g., generated at the comparing operation 104). From here, the at least one electronic processor 20 is programmed to determine whether an unscheduled hematology or histopathology test could be performed on the blood or tissue specimen already required to perform the physician-ordered test to obtain at least a portion of the missing information. Finally, the output operation 106 of the recommendation 40 for the patient to undergo the at least one recommended medical test 38 includes outputting, on the display device 24, a recommendation to perform the unscheduled hematology or histopathology test.

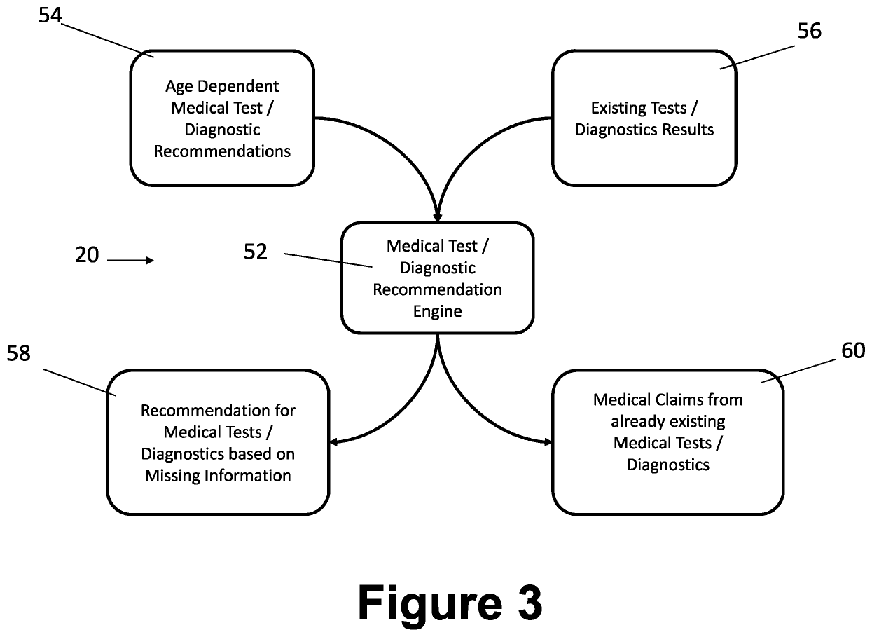

FIG. 3 shows a visualization of the operation of the at least one electronic processor 20. As shown in FIG. 3, and described previously, the at least one electronic processor 20 is programmed as a recommender engine 52 to output notifications 40 for the recommended medical test(s) (or diagnostic(s)) 38. The recommender engine 52 receives, as input, information on age-dependent medical tests 54 (e.g., from the guidelines 34 of the medical tests 36), and the information 56 mined from the patient data retrieved from the database 32. From the inputs 54 and 56, the recommender engine 52 is programmed to execute a matching algorithm to generate an output 58 including the recommendations 40 for the medical test(s) or diagnostic(s) 38, along with determining any missing information needed to complete the recommended tests. The recommender engine 52 is also programmed to generate an output 60 of medical claims from existing medical tests without requiring additional information.

The matching algorithm includes using a set of recommended age-related medical test or diagnostics M (including a subset of medical test and/or diagnostics Mᵢ that are necessary to make a medical claim.), along with a per patient set of existing results of medical tests/diagnostics R. For each recommended, age related medical test/diagnostics Mᵢ, the complement Cᵢ of Mᵢ with respect to R, according to Cᵢ=Mᵢ\R, which outputs a set of additional medical tests/diagnostics that are necessary to complete the information needed for Mᵢ. This way, only the missing medical tests/diagnostics need to be performed, which results in lower effort and costs. Additionally, by calculating Cᵢ for all Mᵢ, the set Mᵢ for which Cᵢ is empty provides those Mᵢ, i.e. those medical claims that can already be made from the existing medical tests/diagnostics at no additional costs and effort.

Another example of the method 100 is described below. A patient has, for example reached an age where a certain cancer screening is recommended by guidelines 34 of the medical tests 36. The cancer screening requires some background information about previous cases of the disease in the family to be correlated with some information from a blood sample. The patient had a blood sample previously taken for another medical test two months ago. The data from that previous test are available to the medical test recommendation engine 52. The engine 52, connected to a software application (“app”) on a user device of the patient (e.g., a smartphone), can now propose to acquire the additional information needed to conclude the screening test either directly through the app or via the patient's doctor at the next regular appointment.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An apparatus for recommending medical tests for a patient, the apparatus including:
at least one electronic processor programmed to:
retrieve patient data stored in a database;
compare the patient data with guidelines for a plurality of medical tests and identify at least one recommended medical test of the plurality of medical tests based on the comparison; and
output, on at least one display device, a recommendation for the patient to undergo the at least one recommended medical test.

2. The apparatus of claim 1, wherein the at least one electronic processor is programmed to:
determine missing information that is missing from the patient data and that, if available, would enable execution of the at least one recommended medical test;
wherein the output of the recommendation for the patient to undergo the at least one recommended medical test includes outputting, on the display device, an identification of the missing information that, if available, would enable execution of the at least one recommended medical test.

3. The apparatus of claim 1, wherein the at least one electronic processor is programmed to:
compare the patient data with guidelines for the plurality of medical tests;
identify an executable medical test of the plurality of medical tests based on the comparison for which the available information is sufficient to perform the executable medical test;
perform the executable medical test using the available information to generate a test result; and
output, on the at least one display device, the test result.

4. The apparatus of claim 3, wherein the at least one electronic processor is programmed to:
determine supporting information that is missing from the patient data and that, if available, would increase confidence in the test result;
wherein the output of the test result includes a recommendation to obtain the supporting information.

5. The apparatus of claim 1, wherein the at least one recommended medical test includes multiple recommended medical tests, and the at least one electronic processor is programmed to:
rank the multiple recommended medical tests based on one or more ranking criteria.

6. The apparatus of claim 5, wherein the at least one electronic processor is programmed to rank the multiple recommended medical tests by operations including:
identifying a ranking criterion of a clinical guideline for each of the recommended tests; and
generating a score for each of the multiple recommended medical tests based on the clinical guideline for each test.

7. The apparatus of claim 5, wherein the at least one electronic processor is programmed to rank the multiple recommended tests by operations including:
identifying a ranking criterion of pre-conditions or characteristics of the patient identified from the retrieved patient data; and
generating a score for each of the multiple recommended medical tests based on the pre-conditions or characteristics of the patient.

8. The apparatus of claim 5, wherein the at least one electronic processor is programmed to rank the multiple recommended tests by operations including:
identifying a ranking criterion of additional lab work required to perform each of the multiple recommended medical tests based on the retrieved patient data; and
generating a score for each of the multiple recommended medical tests based on the additional lab work required for each test.

9. The apparatus of claim 5, wherein the at least one electronic processor is programmed to rank the recommended tests by operations including:
identifying a ranking criterion of an insurance reimbursement level for each test based on the retrieved patient data; and
generating a score for the multiple recommended tests based on the insurance reimbursement level for each test.

10. The apparatus of claim 5, wherein the at least one electronic processor is programmed to rank the multiple recommended tests by operations including:
identifying a ranking criterion of a patient already having undergone one of the tests based on the retrieved patient data; and
generating a score for the multiple recommended tests based on the patient already having undergone any of the tests.

11. The apparatus of claim 1, wherein the output includes:
generating a user interface showing at least one of: the recommendation of the at least one recommended medical test, a scope of the at least one recommended medical test, a level of detail for the at least one recommended medical test, currently available patient data stored in the database, and additional data needed to complete the at least one recommended medical test; and
transmitting at least the recommendation to a physician.

12. The apparatus of claim 11, wherein the at least one electronic processor is programmed to:
receive an input from the physician indicating rejection of at least one of the recommended medical tests shown on the user interface; and
update the user interface to remove any rejected test.

13. The apparatus of claim 1, wherein the output includes:
generating a user interface showing at least one of: the recommendation of the recommended at least one recommended medical test, currently available patient data stored in the electronic medical record database, and additional data needed to complete the at least one recommended medical test; and
transmitting at least the recommendation to a patient or an insurance company of the patient.

14. The apparatus of claim 1, wherein the retrieval of the patient data stored in the database includes:
determining an acquisition date of items of the patient data stored in the database; and
remove any item of the patient data having an acquisition date that is older than a predetermined time threshold.

15. The apparatus of claim 1, comprising a laboratory procedure scheduling system, and the at least one electronic processor is further programmed to:
schedule a physician-ordered laboratory procedure for collecting a blood or tissue specimen from the patient;
determine missing information that is missing from the patient data and that, if available, would enable execution of the at least one recommended medical test;
determine an unscheduled hematology or histopathology test that could be performed on the blood or tissue specimen to obtain at least a portion of the missing information;
wherein the output of the recommendation for the patient to undergo the at least one recommended medical test includes outputting, on the display device, a recommendation to perform the unscheduled hematology or histopathology test.

16. A non-transitory computer readable medium storing instructions executable by at least one electronic processor to perform a method of recommending medical tests for a patient, the method including:
retrieving patient data stored in a database;
comparing the patient data with guidelines for a plurality of medical tests;
identifying multiple recommended medical tests of the plurality of medical tests based on the comparison;
ranking the multiple recommended medical tests based on one or more ranking criteria; and
outputting, on at least one display device, a list of multiple recommendations for the patient to undergo the multiple recommended medical tests according to the one or more ranking criteria.

17. The non-transitory computer readable medium of claim 16, wherein the method further includes:
determining missing information that is missing from the patient data and that, if available, would enable execution of the at least one recommended medical test; and
outputting, on the display device, an identification of the missing information that, if available, would enable execution of the at least one recommended medical test.

18. The non-transitory computer readable medium of claim 17, wherein the method further includes:
comparing the patient data with guidelines for the plurality of medical tests; and
identifying an executable medical test of the plurality of medical tests based on the comparison for which the available information is sufficient to perform the executable medical test;
performing the executable medical test using the available information to generate a test result; and
outputting, on the at least one display device, the test result.

19. A method for recommending medical tests for a patient, the method including:
retrieving patient data stored in a database;
comparing the patient data with guidelines for a plurality of medical tests;
identifying at least one recommended medical test of the plurality of medical tests based on the comparison;
generating a user interface showing at least one of: a recommendation of the at least one recommended medical test, currently available patient data stored in the electronic medical record database, and additional data needed to complete the at least one recommended medical test; and
transmitting at least the recommendation to one or more of the patient, a doctor of the patient, and an insurance company of the patient.

20. The method of claim 19, further including:
receiving an input from the physician indicating rejection of at least one of the recommended medical tests shown on the user interface; and
updating the user interface to remove any rejected test.
